(19) United States
(12) Patent Application Publication
(10) Pub. No.: US 2021/0004482 A1
(43) Pub. Date: Jan. 7, 2021

(54) SYSTEM AND METHOD OF ENHANCING SECURITY OF DATA IN A HEALTH CARE NETWORK

(71) Applicant: Patientory, Inc., Atlanta, GA (US)

(72) Inventor: Chrissa Tanelia McFarlane, Atlanta, GA (US)

(21) Appl. No.: 16/584,605

(22) Filed: Sep. 26, 2019

Related U.S. Application Data
(60) Provisional application No. 62/737,026, filed on Sep. 26, 2018.

(51) Int. Cl. G06F 21/62; G16H 10/60

(52) U.S. Cl. CPC G06F 21/6245; G16H 80/00; G06F 21/6227; G16H 10/60

(57) ABSTRACT

A system and a method of enhancing security of data in a health care network are described. The method includes providing a Health Information Exchange (HIE) server implemented over the blockchain to store users' health information and providing a user device present in communication with the HIE server. Events of accessing a user's health information may be monitored using an Artificial Intelligence (AI) learning module to determine a typical access pattern. All access requests may be compared with the typical access pattern to determine an unusual access behavior corresponding to malicious attempts for breach of the user's health information. The user may be reported about such unusual access behavior to enhance the security of data.

### OTHER RELATED APPLICATIONS

The present application is a U.S. Non-Provisional patent application claiming priority of U.S. Provisional Patent Application Ser. No. 62/737,026 filed on Sep. 26, 2018, which is hereby incorporated by reference.

### BACKGROUND OF THE INVENTION

### 1. Field of the Invention

The present disclosure is generally related to data processing, and more particularly related to a method of enhancing security of data stored over a blockchain.

### 2. Description of the Related Art

The subject matter discussed in the background section should not be assumed to be prior art merely because of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

The subject matter discussed in the background section should not be assumed to be prior art merely because of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information, the information may be stored in an encrypted form. Blockchain leverages both cloud networks and encryption to define storage of all information in a block wise manner. The blocks are added to the blockchain in a linear and chronological order. The blockchain helps to store and track data in a secured manner. The blockchain may be used in various fields such as gaming and gambling, diamond industry, real estate, medical industry, or e-voting.

Utilizing blockchain technology in various fields involves multi-parties accessing patient information when authorized to examine healthcare specific behaviors, such as the type of provider accessing the information. These behaviors are examined to identify anomalous behavior of users. It is important to enhance security of blockchain database by monitoring data breach and unusual activity pertaining to user's data.

Applicant believes that a related reference corresponds to U.S. Pat. No. 7,805,377B2 issued for a method for controlling access to a medical record of a patient hosted by at least one medical record repository, comprising a plurality of sub-records, each sub-record having an associated different patient-controlled access control criterion. However, the reference differs from the present invention because it fails to address the issue of enhancing security of a blockchain database by monitoring data breach and unusual activity pertaining to a user's data. The present invention addresses this issue by providing a database utilizing blockchain to enhance its security.

Therefore, there is a need for a system and method effectively and efficiently monitor malicious behavior and breaches of the data to secure access points of the blockchain database.

### SUMMARY OF THE INVENTION

It is one of the objects of the present invention to provide a system and method of enhancing security of data in a health care network that utilizes block chain to store and track data in a secured manner.

It is another object of this invention to provide a system and method of enhancing security of data in a health care network that enhances the security of a blockchain database by monitoring data breach and unusual activity pertaining to a user's data.

It is still another object of the present invention to provide a system and method of enhancing security of data in a health care network to determine unusual access behavior corresponding to malicious attempts for breach of the user's health information.

It is yet another object of the present invention to provide a system and method of enhancing security of data in a health care network that notifies a user of unusual access behavior regarding their health information through a user device.

It is yet another object of the present invention to provide a system and method of enhancing security of data in a health care network for monitoring the access events of a user's health information using artificial intelligence, such that the system continues to learn and enhances the security of the system.

It is another object of this invention to provide such a device that is inexpensive to implement and maintain while retaining its effectiveness.

Further objects of the invention will be brought out in the following part of the specification, wherein detailed description is for the purpose of fully disclosing the invention without placing limitations thereon.

### BRIEF DESCRIPTION OF THE DRAWINGS

The above and other related objects in view, the invention consists in the details of construction and combination of parts as will be more fully understood from the following description, when read in conjunction with the accompanying drawings in which:

FIG. 1 illustrates a network connection diagram of a Health Information Exchange (HIE) system for enhancing security of data in a health care network, according to various embodiments.

FIG. 2 illustrates a method for symmetric encryption of data, according to various embodiments.

FIG. 2A illustrates a method for asymmetric encryption of data, according to various embodiments.

FIG. 3 illustrates a method for hybrid encryption of data, according to various embodiments.

FIG. 4 illustrates a system for storing and accessing data in a health care network, according to various embodiments.

FIG. 5 illustrates a system for storing and accessing data in a health care network implemented over a blockchain network, according to various embodiments.

FIG. 6 illustrates a flowchart showing a method performed by an approver module, according to various embodiments.

FIG. 7 illustrates a flowchart showing a method performed by a securer module, according to various embodiments.

FIG. 8 illustrates a flowchart showing a method performed by an alerter module, according to various embodiments.

FIG. 9 illustrates a flowchart showing a method performed by an Artificial Intelligence (AI) learning module, according to various embodiments.

### DETAILED DESCRIPTION OF THE EMBODIMENTS OF THE INVENTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of various embodiments of the present disclosure, various embodiments of the systems and methods will be described.

Embodiments of the present disclosure will be described more fully hereinafter with reference to the accompanying drawings in which like numerals may represent like elements throughout the several figures, and in which various example embodiments are shown. Various embodiments may, however, be embodied in many different forms and should not be construed as limited to the embodiments set forth herein. The examples set forth herein are non-limiting examples and are merely examples among other possible examples.

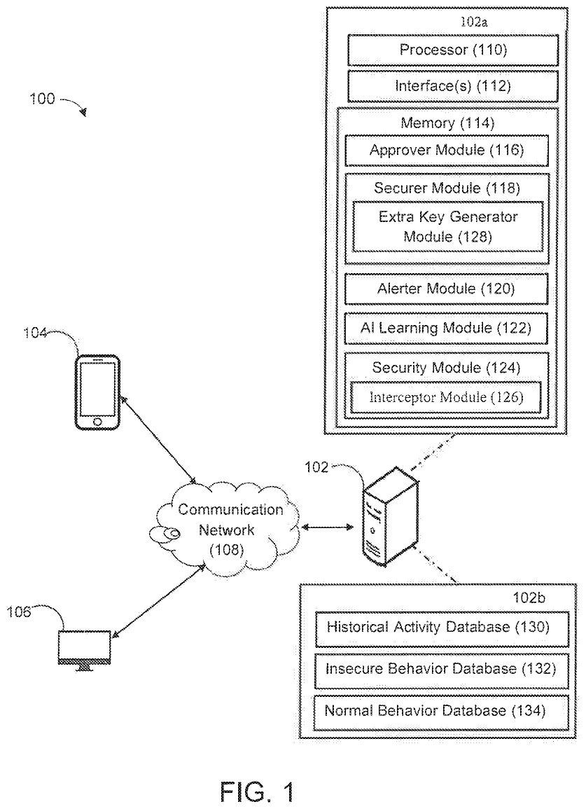

FIG. 1 illustrates a network connection diagram 100 of a Health Information Exchange (HIE) system 102 for enhancing security of data in a health care network. The HIE system 102 may comprise one or more user interfaces. The one or more user interfaces may be accessed by one or more user via one or more devices. The one or more device may comprise, for example, a user device 104 and a health care support device 106. The HIE system 102 may be connected with the user device 104 and the health care support device 106, through a communication network 108.

The communication network 108 may be a wired and/or a wireless network. The communication network 108, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The HIE system 102 may comprise a group of components 102a for enhancing the security of the blockchain database over the health care network. The group of components 102a may include a processor 110, interface(s) 112, and a memory 114. The memory 114 may include an approver module 116, a securer module 118, an alerter module 120, an Artificial Intelligence (AI) learning module 122, and a security module 124. Further, the securer module 118 may include an interceptor module 126. The security module 124 may include an extra key generator module 128. Further, the HIE system 102 may comprise a group of components 102b which may include a historical activity database 130, an insecure behavior database 132, and a normal behavior database 134. It should be noted that the approver module 116, the securer module 118, the alerter module 120, the AI learning module 122, the security module 124, the interceptor module 126, the extra key generator module 128, the historical activity database 130, the insecure behavior database 132, and the normal behavior database 134 may be a part of a storage module (not shown).

Further, the processor 110 may execute an algorithm stored in the memory 114 for enhancing security of data in a health care network. The processor 110 may also be configured to decode and execute any instructions received from one or more other electronic devices or server(s). The processor 110 may include one or more general purpose processors (e.g., microprocessors) and/or one or more special purpose processors (e.g., digital signal processors (DSPs), System On Chips (SOCs), Field Programmable Gate Arrays (FPGAs)). The processor 110 may be configured to execute one or more computer-readable program instructions, such as program instructions to carry out any of the functions described in this description.

The interface(s) 112 may help an operator to interact with the HIE system 102. The interface(s) 112 may either accept inputs from users or provide outputs to the users, or may perform both the actions. In various embodiments, a user can interact with the interface(s) 112 using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or any combination of the above. Further, the interface(s) 112 may either be implemented as a Command Line Interface (CLI), a Graphical User Interface (GUI), a voice interface, or a web-based user-interface.

The memory 114 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions. The memory 114 may comprise modules implemented as a program. As described above, the memory 114 may comprise the approver module 116, the securer module 118, the alerter module 120, the AI learning module 122, and the security module 124. The securer module 118 may include the interceptor module 126. The security module 124 may include the extra key generator module 128.

In various embodiments, several users may interact with the HIE system 102, using the user device 104. Although a single user device has been illustrated, several user devices could similarly be connected to the communication network 108. Further, each of the user devices may have a device ID. In various embodiments, the device ID may be a unique identification code such as an (International Mobile Equipment Identity) IMEI code or a product serial number. It should be noted that a user may use a single user device or multiple user devices. Further, multiple users may use a single user device or multiple user devices. Further, the one or more users may receive and/or provide healthcare related products and services. The one or more users may include, for example and not limited to, patients, family and friends of the patients, hospitals, physicians, nurses, specialists, pharmacies, medical laboratories, testing centers, insurance companies, or Emergency Medical Technician (EMT) services.

The user device 104 may be a stationary device, a portable device, or a device accessed remotely. The user device 104 may be, but not limited to, a computer, a laptop, a tablet, a mobile phone, a smartphone, an Internet of Things (IoT) device, or a smart watch. In various embodiments, the user device 104 may include an imaging device that may be configured to capture a visual graphical element. The visual graphical element may be, for example but not limited to, a barcode, text, a picture, or any other forms of graphical authentication indicia. In various embodiments, the barcode may be one-dimensional or two-dimensional. Further, the imaging device may include a hardware and/or software element. In various embodiments, the imaging device may be a hardware camera sensor that may be operably coupled to the user device 104. In various embodiments, the hardware camera sensor may be embedded in the user device 104. In various embodiments, the imaging device may be located external to the user device 104. In various embodiments, the imaging device may be connected to the user device 104 wirelessly or via a cable. It should be noted that image data of the visual graphical element may be transmitted to the user device 104 via the communication network 108. In various embodiments, the imaging device may be controlled by applications and/or software(s) configured to scan a visual graphical code. In various embodiments, a camera may be configured to scan a QR code. Further, the applications and/or software(s) may be configured to activate the camera present in the user device 104 to scan the QR code. In various embodiments, the camera may be controlled by a processor natively embedded in the user device 104. In various embodiments, the imaging device may include a screen capturing software (for example, screenshot) that may be configured to capture and/or scan the QR code on a screen of the user device 104.

In various embodiments, the user device 104 may collect information related to the user's daily health status. The user device 104 may be used to accept input(s) from user for creating a user account. In various embodiments, the user may provide personal identification information, for example a telephone number or an email address, to create the user account. It should be noted that personal information entered by the user is stored in one or more databases. After creating the user account, the HIE system 102 may create a digital wallet (e.g., a PTOYNet Ethereum wallet) and private key for the user. In various embodiments, the private key stored by the HIE system 102 on the user's device 104 may be used for identification and authorization by the user. Public keys corresponding to the private keys may be stored by the HIE system 102 on one or more databases.

In various embodiments, a group of databases 102b may be connected to the HIE system 102. In various embodiments, the group of databases 102b may be implemented over a blockchain network (such as a PTOYNet blockchain network or a PTOYNet Ethereum™ Blockchain network), and may be present as different databases installed at different locations. The group of databases 102b may comprise the historical activity database 130, the insecure behavior database 132, and the normal behavior database 134. The group of databases 102b may be configured to store data belonging to different users and data required for functioning of the HIE system 102. Different databases can be used in accordance with various embodiments; however, a single database may also be used for storing the data. Usage of the different databases may also allow segregated storage of different data and may thus reduce time to access desired data. In various embodiments, the data may be encrypted, time-dependent, piece-wise, and may be present as subsets of data belonging to each user. In various embodiments, the data may represent the results of one medical test in a series of multiple medical tests.

In various embodiments, the group of databases 102b may operate collectively or individually. Further, the group of databases 102b may store data as tables, objects, or other data structures. Further, the group of databases 102b may be configured to store data retrieved or processed by the HIE system 102. The data may include, but not limited to, a patient medical history, medical charts, medications, prescriptions, immunizations, test results, allergies, insurance provider, or billing information. Further, the data may be time-dependent and piece-wise. Further, the data may represent a subset of data for each patient. In various embodiments, the data may represent results of a medical test in a series of multiple medical tests. Further, the data may be securely stored. In various embodiments, the data may be encrypted.

In various embodiments, information stored in the group of databases 102b may be accessed based on users' identities and/or the users' authorities. The users' identities may be verified in one or more ways such as, but not limited to, biometric authentication (or bio authentication), password or PIN information, user device registrations, a second-level authentication, or a third-level authentication. In various embodiments, the users' identities may be verified by the HIE system 102. Information provided by the users in a real-time may be used, by the HIE system 102, to confirm the users' identities. In an example, the users' identities may be verified using a name, a password, one or security questions, or any combination thereof. In various embodiments, a user may be identified using an encryption key and/or a decryption key.

In various embodiments, the data stored in the group of databases 102b may be accessed at different levels, for example using a first level subsystem and a second level subsystem. In various embodiments, a user may directly access the first level subsystem. To access data stored in the second level subsystem, the second level subsystem may need to be accessed through the first level subsystem. It should be noted that the communication between the first level subsystem and the second level subsystem may be encrypted. In various embodiments, the second level subsystem may be implemented over a blockchain network (such as a PTOYNet blockchain network). In various embodiments, the PTOYNet blockchain network may be used to implement smart contracts.

In various embodiments, a primary care physician may input data into the HIE system 102 using the user device 104. The data may be processed by the first level subsystem and the second level subsystem. This may be done successively. The data may be stored on the first level subsystem and/or the second level subsystem of the HIE system 102. This may be done successively. The data may include, but not limited to, one or more instructions to a patient to see a physician specialist. Further, the data may be stored in one or more blockchains of the second level subsystem. The patient may be able to access the data relating to the patient's care provided by the primary care physician. This may be done successively. The patient may be able to retrieve the data using the user device 104 of the patient. This may be done successively.

In accordance with various embodiments, the patient may communicate with the physician specialist using the HIE system 102. It should be noted that the physician specialist may be able to access the data of the patient from the first level subsystem and/or the second level subsystem. Further, the physician specialist may be able to communicate with the patient. It should be noted that some, all (or substantially all) communications between the primary care physician, the physician specialist and the patient may be stored and may be accessible on a blockchain network.

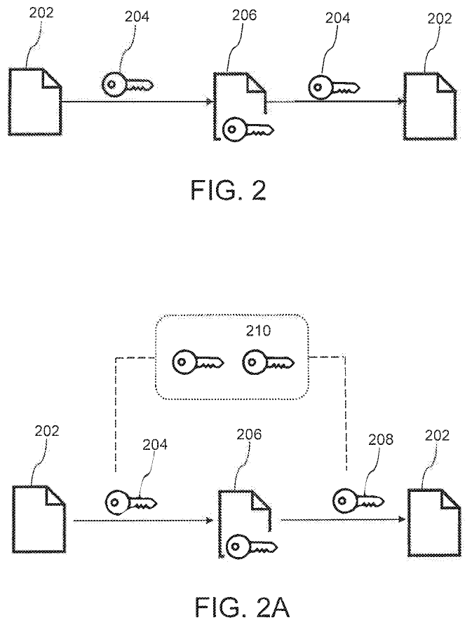

FIG. 2 illustrates a method for symmetric encryption of data, according to various embodiments. Original data 202 may be encrypted using a key 204 to obtain an encrypted data 206. The encrypted data 206 may be decrypted using the key 204 to obtain back the original data 202. It should be noted that encryption and decryption of the data may be performed using a same key. Further, one or more parties involved in a communication may have the same key to encrypt and decrypt the data.

FIG. 2A illustrates a method for asymmetric encryption of data, according to various embodiments. Original data 202 may be encrypted using a key 204 to obtain encrypted data 206. Encrypted data 206 may be decrypted using another key 208 to obtain the original data 202. It should be noted that encryption and decryption of the data may be performed using different keys e.g., a key pair 210.

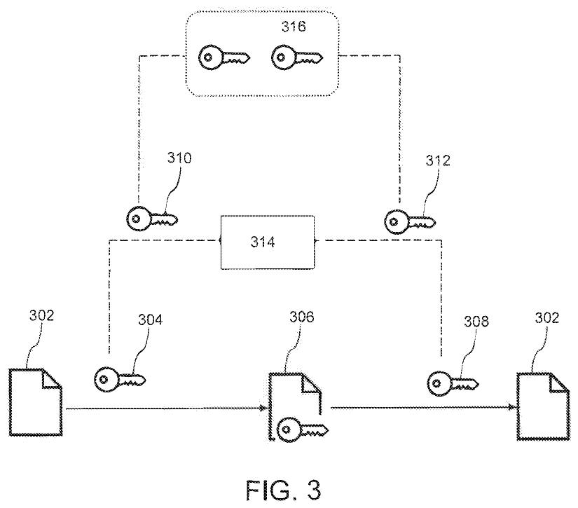

FIG. 3 illustrates a method for hybrid encryption of data, according to various embodiments. Symmetric encryption and asymmetric encryption techniques may be used in tandem. In various embodiments, the symmetric encryption technique may be used to encrypt data 302 using a symmetric key 304 for producing encrypted data 306. The encrypted data 306 may be decrypted using another symmetric key 308 for obtaining data 302 (e.g., back data). Further, a public key 310 may be used to encrypt the symmetric key 304 and a private key 312 may be used to encrypt the symmetric key 308, stored as an encrypted key 314. The public key 310 and the private key 312 may form a key pair 316.

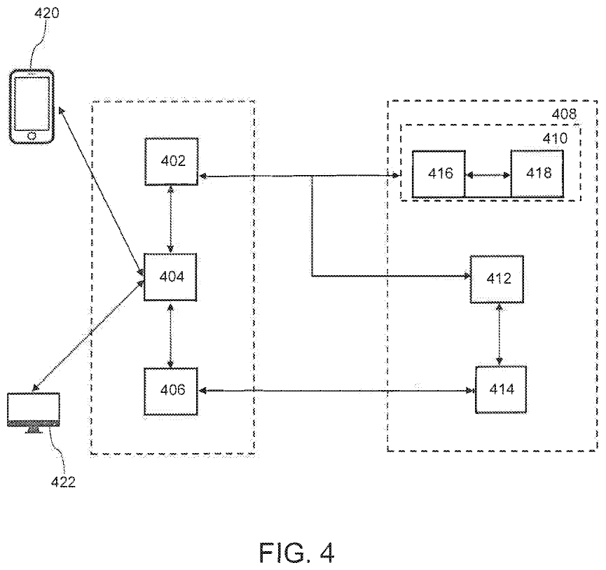

In accordance with various embodiments, referring to FIG. 4 illustrating an example of a system for storing and accessing data in a health care network, the first level subsystem may include a core service component 402 and a Remote Procedure Call (RPC) component 404. In various embodiments, the second level subsystem may include a quorum blockchain node component 406. In various embodiments, the first level subsystem may include the core service component 402, and the second level subsystem may include the RPC component 404 and the quorum blockchain node component 406. Further, the core service component 402 of the first level subsystem may be present in communication with third-party servers and databases of a hospital computing network 408. The hospital computing network 408 may include an Interplanetary File System (IPFS) module 410, an EHR synchronization service 412, and a quorum blockchain node 414. Further, the IPFS module 410 may include an IPFS manager 416 and an IPFS node 418. The quorum blockchain node component 406 of the second level subsystem may communicate with the quorum blockchain node 414 of the hospital computing network 408. Patients may access the health care network for storing data through a user device 420, and a representative of a hospital may access the health care network through another user device 422.

In accordance with various embodiments, the core service component 402 may be referred as software module that can communicate with third-party servers and databases. The core service component 402 may be in communication with, for example, the servers and databases of a hospital computing network. The RPC component 404 may communicate signed communication from the user device 104 to quorum blockchain node 414. In various embodiments, the signed transaction may grant permission to a hospital representative to view the data. The quorum blockchain node 402 may activate one or more smart contracts post confirming ownership of the signed transaction. In various embodiments, the confirmation may be granted once the RPC component 404 communicates the signed transaction to the quorum blockchain node 414. The quorum blockchain node 402 may contain a hash for patient blockchain. In various embodiments, the quorum blockchain node 402 may revise a state of one or more blockchains.

In accordance with various embodiments, the representative of the hospital may want to synchronize Electronic Health Record (EHR) data of a patient. The first level subsystem and the second level subsystem may ask the patient for permission to allow a representative of the hospital to store the EHR data of the patient, through the IPFS module 410. This may be done successively. Based at least on the permission granted by the patient, a signed transaction may be created to confirm the permission of the hospital to store the EHR data. Further, the signed transaction may activate a smart contract that may add hospital identification information such as a blockchain address to a list of permitted users.

In accordance with various embodiments, the signed transaction may be transmitted from the user device to the RPC component 404 of the first level subsystem and/or the second level subsystem. The RPC component 404 may communicate the signed transaction to the quorum blockchain node component 406 of the second level subsystem. This may be done successively. The quorum blockchain node component 406 may activate one or more smart contracts. Thereafter, the quorum blockchain node component 406 may revise a state of one or more blockchains. This may be done successively.

In accordance with various embodiments, based at least on the permission granted by the patient, the EHR synchronization service may obtain a list of patients from the RPC component 404. The EHR synchronization service may confirm whether the patient has granted permission. Based at least on the permission, the first level subsystem and the second level subsystem may obtain the EHR data and may calculate a hash function for the EHR data. The HIE system 102 may match the hash function of the EHR data with a hash function for the patient blockchain on the quorum blockchain node component 406 of the second level subsystem. This may be done successively. If the hash function of the EHR data matches with the hash function for the patient blockchain on the quorum blockchain node component 406 of the second level subsystem, the EHR data of the patient may remain unchanged.

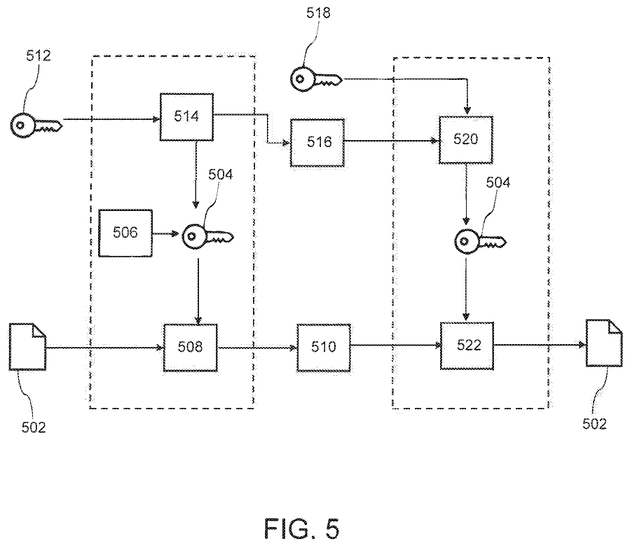

In accordance with various embodiments, referring to FIG. 5 illustrating a system for storing and accessing data in a health care network implemented specifically over a blockchain network (such as a PTOYNet blockchain network or a PTOYNet Ethereum™ Blockchain network), the HIE system 102 may execute an application for determining permission from the user for obtaining EHR data 502. In various embodiments, if the user grants the permission, the HIE system 102 may obtain the EHR data 502 for calculating a hash function for the EHR data 502. The HIE system 102 may match the hash function of the EHR data 502 with a hash function for the user blockchain on the quorum blockchain node of the second level sub-system. In various embodiments, if the two hash matches, there is no change to the user's EHR data 502. In various embodiments, the two hash functions do not match, the HIE system 102 may generate a random string e.g., secret key 504, through a random key generator 506. The secret key 504 may be used for Advanced Encryption Standard (AES) encryption of the EHR data 502, in an AES encryptor 508, for generating encrypted EHR data 510.

In accordance with various embodiments, the secret key 504 may then be encrypted by, for example, a Rivest-Shamir-Adleman (RSA) public key 512 of the patient, in an RSA encryptor 514, to generate an encrypted secret key 516. The HIE system 102 may also send the encrypted EHR data 510 to the core service component 402 for forwarding the data to the IPFS manager 416 of the hospital computing network 408 for storage. The IPFS manager 416 may send an IPFS hash function to the core service component 402 for further sending the IPFS hash function to EHR synchronization service 412. The EHR synchronization service 412 may further update the patient smart contract with the new IPFS hash function, the encrypted random key, a hash function of the unencrypted file, and file name.

In accordance with various embodiments, a hospital representative, such as a doctor or a hospital administration, may want to view the EHR data 502. In such a scenario, the user may first send a signed transaction to a RPC component 404 for granting permission to the hospital representative to view the EHR data 502. Once the permission is granted, the signed transaction may be added to the quorum blockchain node 414 and a new smart contract will be created for a blockchain corresponding to the hospital representative. After adding the signed transaction, the hospital representative may be able to view the EHR data 502 of the user, on a device.

In various embodiments, in order to view the EHR data 502 on the device, the HIE system 102 may collect the encrypted EHR data 510 from the user's blockchain and may decrypt the encrypted EHR data 510 using patient's RSA private key 518. The HIE system 102 may decrypt the encrypted secret key 516, in an RSA decryptor 520, using RSA private key of the hospital representative. The encrypted EHR data 510 may be decrypted using the RSA public key 512 of the hospital representative, in an AES decryptor 522. This may be done successively. Further, the HIE system 102 may load the decrypted EHR data 502 to the smart contract previously created for the hospital representative.

After loading the decrypted EHR data 502, the RPC component 404 may obtain the signed transaction from the patient's user device and transmit the signed transaction to the quorum blockchain node component 406 of the second level subsystem. The quorum blockchain node component 406 may confirm ownership of the signed transaction and may execute the smart contract for the hospital representative to view the user's data.

In various embodiments, the patient may decline permission for the hospital representative to have access to the EHR data 502. In such an example scenario, the user through a user device may send a signed transaction revoking permission to the RPC component 404. The RPC component 404 may forward the signed transaction to the quorum blockchain node component 406 of the second level subsystem. The quorum blockchain node component 406 may confirm ownership of the signed transaction and may delete the smart contract previously created to allow the hospital representative to have access to the patient's EHR data 502.

In accordance with various embodiments, the HIE system 102 may comprise a health record network for an intermediary enabling sharing of user's medical records with providers. For enabling sharing, the user may grant specific permissions to the providers for accessing parts of the user's medical records stored in one or more databases implemented over a blockchain network. The user may also grant specific permissions to modify the user's medical records in the one or more databases. In various embodiments, the user may include, for example, any users constituting a value chain, such as doctors, nurses etc. In various embodiments, the user may be doctors remotely logging into the HIE system 102 or doctors present in hospitals.

In various embodiments, the HIE system 102 may be connected with the health care support device 106, through the communication network 108. The health care support device 106 may receive some type of patient data or user's health information. The user's health information may include blood pressure, heart rate, and number of steps moved per day. Further, the health care support device 106 may be operated by doctors, hospitals, physicians, nurses, specialists, pharmacies, medical laboratories and testing centers, insurance companies, or Emergency medical technician (EMT) services. Although a single health care support device has been illustrated, several health care support devices could similarly be connected to the communication network 108. The health care support device 106 may be a stationary device, a portable device, or a remote device. The health care support device 106 may be, for example but not limited to, a computer, a laptop, a tablet, a mobile phone, a smartphone, an Internet of Things (IoT) device, or a smart watch.

In various embodiments, the historical activity database 130 may be configured to store activities of RPC component 404 requests for building a complete profile of an entity's RPC component 404 that requests behavior for comparison. In various embodiments, the insecure behavior database 132 may be configured to store criteria showing insecure blockchain access behavior, such as requests to multiple unrelated blocks, remaining logged in with a private key too long, or suspected spoofing. In various embodiments, the normal behavior database 134 may be configured to store criteria showing normal and secure access behavior, such as normal usage of the blockchain data.

In various embodiments, the security module 124 may include the interceptor module 126 that may receive all requests from the RPC component 404 before the requests are sent to the quorum blockchain node 402. Further, the interceptor module 126 may use well known means to alert for cyber security threats such as leveraging threat intelligence. In various embodiments, the threat intelligence may refer to a way of looking at signature data from previously seen attacks and comparing the signature data to enterprise data to identify threats. The threat intelligence may be used to a great effect in, for example, Security Information and Event Management (STEM), antivirus, Intrusion Detection System (IDS), and web proxy technologies.

In various embodiments, the interceptor module 126 may use well known means to alert for cyber security threats such as analyzing user and attacker behavior analytics. In the user behavior analytics, an organization may be able to gain a baseline understanding of what normal behavior for an employee, what kind of data the employee may access, what times the employee log on, and where the employee are physically located. In various embodiments, a 2 a.m. logon in Shanghai from someone who usually works from 9 a.m. to 5 p.m. in New York and does not travel for business may stand out as unusual behavior and something a security analyst may need to investigate. In the attacker behavior analytics, no baseline of activity may be present for comparing information to small, seemingly unrelated activities detected on a network over time may in fact be breadcrumbs of activity that an attacker leaves behind. It should be noted that both technology and the human mind may be used to put all pieces together but may help form a picture of what an attacker may be up to within an organization's network.

In various embodiments, the interceptor module 126 may use well known means to alert for cyber security threats such as setting intruder traps. It should be noted that some targets may be tempting for an attacker to pass up. Security teams may set traps so that the attacker may take bait. Within the organization's network, an intruder trap may include, for example, a honeypot target that may seem to house network services especially appealing to an attacker, or honey credentials that appear to have user privileges an attacker may need to gain access to sensitive systems or data. When the attacker goes after the bait, the interceptor module 126 may trigger an alert so that the security team may know whether there is a suspicious activity in the network that needs to be investigated.

In various embodiments, the interceptor module 126 may use well known means to alert for cyber security threats such as conducting threat hunts. The threat hunts may enable security analysts to actively go into the network, endpoints, and security technology to look for threats or attackers that may be lurking as-yet undetected. Such a technique may be performed by veteran security and threat analysts. It should be noted that a well-developed security threat detection program may include all of the above tactics, amongst others, to monitor the security of the organization's employees, data, and critical assets.

In various embodiments, the interceptor module 126 may use well known means to alert for cyber security threats, for example but not limited to, threat detection by a two-pronged approach. In various embodiments, the threat detection may require both a human element and a technical element. The human element may include security analysts who analyze trends, patterns in data, behaviors, and reports. The security analysts may determine if anomalous data indicates a potential threat or a false alarm. In various embodiments, the technical element may include any combination of tools acting as a net across the entirely of an organization's network, from end to end, to try and capture threats before they become a serious problem.

In various embodiments, the interceptor module 126 may use well known means to alert for cyber security threats for employing security event threat detection technology. Such detection technology may aggregate data from events across the network, including authentication, network access, and logs from critical systems, and network threat detection technology. The network threat detection technology may understand traffic patterns on the network and monitor traffic within and between trusted networks, including the internet. In various embodiments, an endpoint threat detection technology may be used to provide detailed information about possibly malicious events on user machines, including any behavioral or forensic information to aid in investigating threats.

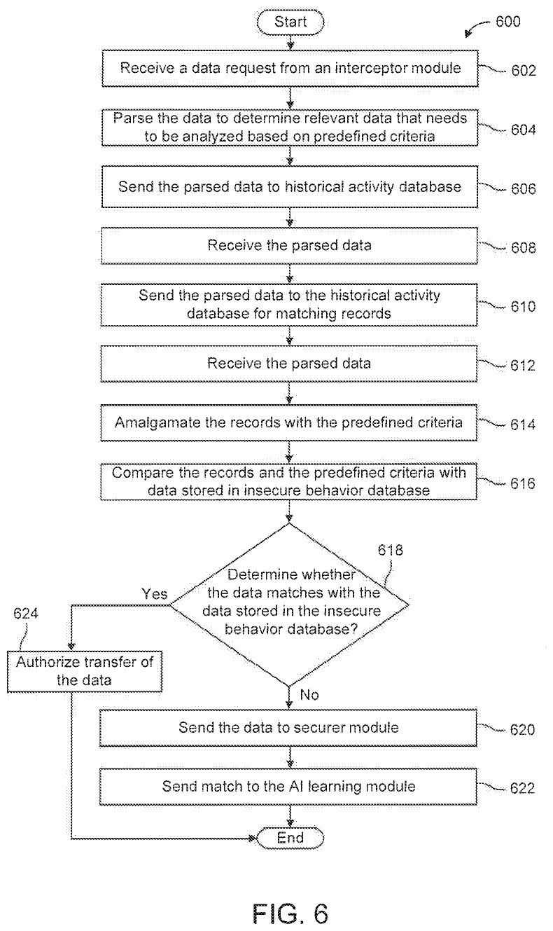

FIG. 6 illustrates an example flowchart 600 showing a method performed by the approver module 116, according to various embodiments. Functioning of the approver module 116 will now be explained with reference to the example flowchart 600 shown in FIG. 6. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The approver module 116 may receive a data request from the interceptor module 126, at step 602. The approver module 116 may parse data to determine relevant data that needs to be analyzed based on predefined criteria, at step 604. This may be done successively. In various embodiments, the predefined criteria may be defined by an inventor or engineer. The approver module 116 may send the parsed data to the historical activity database 130, at step 606. This may be done successively. The approver module 116 may receive the parsed data, at step 608. This may be done successively. In various embodiments, the parsed data may be sent to a historical block write database for storing the parsed data as a reference copy.

The approver module 116 may send the parsed data to the historical activity database 130 for matching records, at step 610. This may be done successively. The approver module 116 may receive the parsed data, at step 612. This may be done successively. The approver module 116 may amalgamate records with the predefined criteria, at step 614. This may be done successively. The approver module 116 may compare the records and the predefined criteria with data stored in the insecure behavior database 132, at step 616. This may be done successively. The approver module 116 may determine whether the data matches with the data stored in the insecure behavior database 132, at step 618. This may be done successively.

In various embodiments, if the data does not match or correlate with the data stored in the insecure behavior database 132, the approver module 116 may send the data to the securer module 118, at step 620. The approver module 116 may send the matches to the AI learning module 122, at step 622. In various embodiments, if the data matches or correlates to the data stored in the insecure behavior database 132, the approver module 116 may authorize transfer of the data, at step 624.

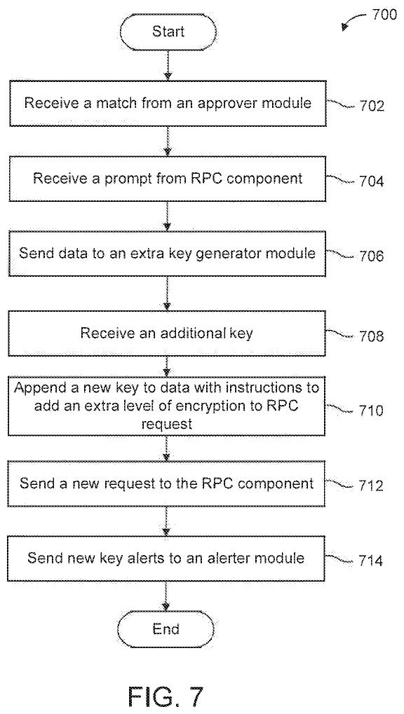

FIG. 7 illustrates an example flowchart 700 showing a method performed by a securer module 118, according to various embodiments. Functioning of the securer module 118 will now be explained with reference to the example flowchart 700 shown in FIG. 7. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The securer module 118 may receive a match from the approver module 116, at step 702. In various embodiments, the match may include the data from the insecure behavior database 132. The securer module 118 may receive a prompt from the RPC component 404, at step 704. This may be done successively. The securer module 118 may send the data to the extra key generator module 128, at step 706. This may be done successively. In various embodiments, the data may be sent for additional keys to be created for the data that may increase the security. It should be noted that the extra key generator module 128 may be a software module for generating encryption tools, such as encryption keys. The securer module 118 may receive the additional key, at step 708. This may be done successively. The securer module 118 may append a new key to the data with instructions to add an extra level of encryption to the RPC request, at step 710. This may be done successively. The securer module 118 may send new request to the RPC component 404, at step 712. This may be done successively. The securer module 118 may send new key alerts to the alerter module 120, at step 714.

In various embodiments, the securer module 118 may generate the new key alerts. It should be noted that the securer module 118 may apply an additional security to the data that has been flagged as having a significant correlation to the insecure behavior database 132.

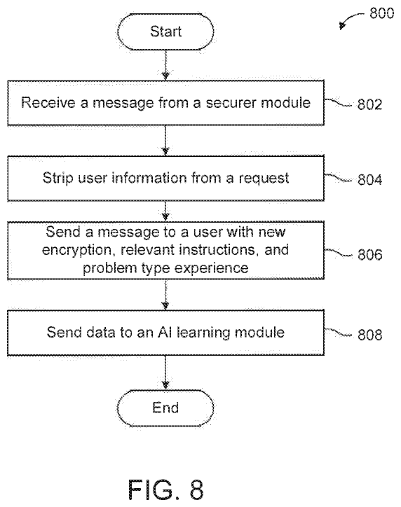

FIG. 8 illustrates an example flowchart 800 showing a method performed by the alerter module 120, according to various embodiments. Functioning of the alerter module 120 will now be explained with reference to the example flowchart 800 shown in FIG. 8. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The alerter module 120 may receive a message from the securer module 118, at step 802. The alerter module 120 may strip user information from a request, at step 804. This may be done successively. In various embodiments, the user information may be stripped to know where to send an alert. The alerter module 120 may send a message to the user with new encryption, relevant instructions, and problem type experience, at step 806. This may be done successively. In various embodiments, the problem type may be too much information trying to be downloaded or too many points of access. The alerter module 120 may send data to the AI learning module 122, at step 808. It should be noted that the data may be sent to further enhance the abilities of the AI learning module 122.

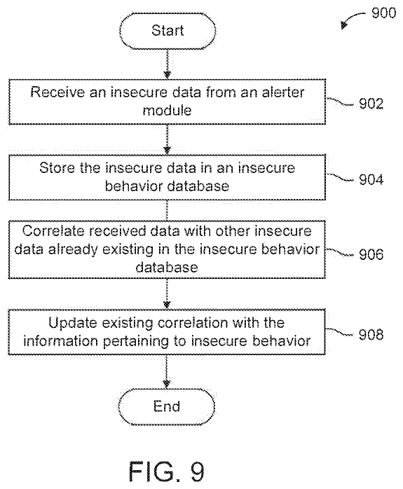

FIG. 9 illustrates an example flowchart 900 showing a method performed by the AI learning module 122, according to various embodiments. Functioning of the AI learning module 122 will now be explained with reference to the example flowchart 900 shown in FIG. 9. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The AI learning module 122 may receive an insecure data from the alerter module 120, at step 902. The AI learning module 122 may store the insecure data in the insecure behavior database 132, at step 904. This may be done successively. In various embodiments, the received data may be stored along with data already stored in the insecure behavior database 132. The AI learning module 122 may correlate the received data with the other insecure data already existing in the insecure behavior database 132, at step 906. This may be done successively. The AI learning module 122 may update existing correlation with the information pertaining to insecure behavior, at step 908.

In various embodiments, the AI learning module 122 may apply machine learning to requests by the RPC component 404 for determining normal or insecure behavior. The AI learning module 122 may receive data of the request from the interceptor module 126. The AI learning module 122 may apply one or more machine learning tools to the data to determine the behavior, which is then stored in a searchable form in an associated database. In various embodiments, one or more machine learning tools may include, for example but not limited to, Association Rule Learning or Neural Networks.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method of enhancing security of data in a health care network, comprising:
a. monitoring, using an artificial intelligence learning module, an access event to a user's health information to determine a typical access pattern, wherein the user's health information is managed by a health information exchange server implemented over a blockchain network;
b. comparing an access request to the user's health information with a typical access pattern to determine an unusual access behavior corresponding to malicious attempts to breach the user's health information; and
c. reporting to the user an unusual access behaviour, via a user device communicatively coupled with the health information exchange server.

2. The computer-implemented method of claim 1, wherein the user's health information comprises blood pressure, heart rate, and number of steps moved per day.

3. The computer-implemented method of claim 1, wherein monitoring an access event to a user's health information comprises parsing a data request to determine a relevant data based on a predefined criterion and sending the relevant data to a historical activity database.

4. The computer-implemented method of claim 1, further comprising comparing the access event with a datum stored in an insecure behavior database and storing the access request in the insecure behavior database when the access event matches the datum at least partially.

5. The computer-implemented method of claim 1, wherein comparing an access request with a typical access pattern comprises providing a datum in the access request to a securer module when the datum has no match in an insecure behavior database.

6. The computer-implemented method of claim 1, further comprising requesting an additional encrypted key when the access request is not a typical access pattern.

7. The computer-implemented method of claim 1, further comprising storing the access request in an insecure behavior database for comparing with a another access request.

8. The computer-implemented method of claim 1, further comprising stripping a user information from the access request and sending an alert with a new encryption to the user based on the user information.

9. The computer-implemented method of claim 1, further comprising modifying the artificial intelligence learning module based when the access event does not fit a typical access pattern.

10. The computer-implemented method of claim 1, further comprising updating an existing correlation with the access request when the access request is identified as an insecure behavior.

11. A system for enhancing security of data in a health care network, comprising:
a. one or more processors; and
b. a memory communicatively coupled to the one or more processors and storing instructions which, when executed by the one or more processors, cause the system to:
monitor, using an artificial intelligence learning module, an access event to a user's health information to determine a typical access pattern, wherein the user's health information is managed by a health information exchange server implemented over a blockchain network;
compare and match an access request to the user's health information with a typical access pattern to determine an unusual access behavior corresponding to malicious attempts to breach the user's health information;
report to the user an unusual access behaviour, via a user device communicatively coupled with the health information exchange server; and
compare and match the access event with a datum stored in an insecure behaviour database, and storing the access request in the insecure behaviour database when the access event matches the datum at least partially.

12. The system of claim 11, wherein to compare an access request with a typical access pattern the one or more processors execute instructions to provide a datum in the access request to a securer module when the datum has no match in an insecure behavior database.

13. The system of claim 11, wherein the one or more processors further execute instructions to request an additional encrypted key when the access request is not a typical access pattern.

14. The system of claim 11, wherein the one or more processors further execute instructions to store the access request in an insecure behavior database for comparing with another access request.

15. The system of claim 11, wherein the one or more processors further execute instructions to strip a user information from the access request and to send an alert with a new encryption to the user based on the user information.

16. The system of claim 11, wherein the one or more processors further execute instructions to modify the artificial intelligence learning module based when the access event does not fit a typical access pattern.

17. The system of claim 11, wherein the one or more processors further execute instructions to update an existing correlation with the access request when the access request is identified as an insecure behavior.

18. A non-transitory, computer readable medium storing instructions which, when executed by a processor, cause a computer to perform a method, the method comprising:
a. monitoring, using an artificial intelligence learning module, an access event to a user's health information to determine a typical access pattern, wherein the user's health information is managed by a health information exchange server implemented over a blockchain network;
b. comparing an access request to the user's health information with a typical access pattern to determine an unusual access behavior corresponding to malicious attempts to breach the user's health information;
c. reporting to the user an unusual access behaviour, via a user device communicatively coupled with the health information exchange server; and
d. comprising comparing the access event with a datum stored in an insecure behaviour database, and storing the access request in the insecure behaviour database when the access event matches the datum at least partially.

19. The non-transitory, computer readable medium of claim 18, wherein monitoring an access event to a user's health information comprises parsing a data request to determine a relevant data based on a predefined criterion and sending the relevant data to a historical activity database.

20. The non-transitory, computer readable medium of claim 18, wherein comparing an access request with a typical access pattern comprises providing a datum in the access request to a securer module when the datum has no match in an insecure behavior database.
